(19) United States
(12) Patent Application Publication
(10) Pub. No.: US 2026/0306633 A1
(43) Pub. Date: Oct. 8, 2026

(54) A COMBINATION THERAPY FOR CANCER TREATMENT

(71) Applicant: Purdue Research Foundation, West Lafayette, IN (US)

(72) Inventor: Meden Isaac-Lam, Michigan City, IN (US)

(21) Appl. No.: 19/481,850

(22) PCT Filed: May 3, 2024
(86) PCT No.: PCT/US2024/027600

§ 371 Date: Nov. 5, 2025

Related U.S. Application Data
(60) Provisional application No. 63/464,303, filed on May 5, 2023.

(51) Int. Cl. A61K 41/00; A61K 31/337; A61K 31/513; A61K 31/519; A61K 31/704; A61K 33/243; A61K 47/54; A61K 47/55; A61P 35/00

(52) U.S. Cl. CPC A61K 41/0071; A61K 31/337; A61K 31/513; A61K 31/519; A61K 31/704; A61K 33/243; A61K 47/546; A61K 47/551; A61P 35/00

(57) ABSTRACT

A pharmaceutical combination comprising (i) a photosensitizing chlorin-vitamin conjugate or a pharmaceutically acceptable salt, hydrate, or metal complex thereof, and (ii) at least one chemotherapeutic agent; a pharmaceutical composition comprising the pharmaceutical combination; a combination of pharmaceutical compositions comprising (i) and (ii); and method of treating cancer.

### CROSS-REFERENCE TO RELATED APPLICATION

This application claims priority to U.S. provisional patent application No. 63/464,303, which was filed May 5, 2023, and which is hereby incorporated by reference in its entirety.

### TECHNICAL FIELD

The present disclosure relates to a combination therapy for the treatment of cancer. In particular, it is a combination of photodynamic therapy and chemotherapy for treating breast cancer using a chlorin-vitamin conjugate and a chemotherapeutic agent.

### BACKGROUND

This section introduces aspects that may help facilitate a better understanding of the disclosure. Accordingly, these statements are to be read in this light and are not to be construed as admissions about what is or is not prior art.

Breast cancer (BC) is the most common type of cancer in women, and the number of new cases in the US is still increasing each year. Triple-negative breast cancer (TNBC), which comprises 15-20% of all breast cancer, is considered the most aggressive type of breast cancer due to lack of estrogen receptor (ER), progesterone receptor (PR), and human epidermal growth factor receptor 2 (HER2) expressions for treatments (Won K A et al., International Journal of Oncology, 2020, 57, 1245-1261). Its complexity and aggressiveness remain a challenge for developing a remedy. TNBC patients do not respond well to existing systemic therapies since TNBC does not express any of the aforementioned biomarkers and shows an increased rate of recurrence.

Traditional chemotherapy is the well-established treatment option for TNBC. Chemotherapy treatment involves the use of drugs to destroy cancer cells. Chemotherapy aims to control cancer, prolong survival, and improve quality of life, but it does not completely eradicate the disease. Non-specificity of the drugs, toxicity, and multi-drug resistance are major drawbacks of chemotherapy. The toxicity of chemotherapeutic drugs, such as hepatotoxicity, cardiotoxicity, renal toxicity, and other systemic effects, limit treatment options available for patients suffering from the disease.

Photodynamic therapy (PDT) for cancer treatment involves the delivery of a light-absorbing component, known as a photosensitizer (PS), to tumor tissues upon systemic administration, followed by visible light (600-800 nm) irradiation in the presence of endogenous oxygen. Excitation of PS in the red or near-infrared (NIR) region produces cytotoxic reactive oxygen species (ROS), such as singlet oxygen (¹O₂), to cause irreversible eradication of tumor cells and induce immune inflammatory responses and damage to tumor vasculature. The immunomodulatory effects of PDT in animal models suggest that it stimulates the host immune system to facilitate the prevention of tumor growth (Banerjee S M et al., 2017, Breast, 35, 105-113). Despite its low toxicity, repeatability, and potential immunological effects, PDT has limitations of PS selectivity and several side effects such as pain, edema, flaking, pustulation, erosion, hyper-hypopigmentation, and infections (Sobhani N et al., 2021, Journal of the Egyptian National Cancer Institute, 33(1), 34).

Thus, there is a need for a therapy that helps to alleviate adverse side effects, address multi-drug resistance, and improve treatment outcomes and quality of life for cancer patients. It is an object of the present disclosure to provide a new cancer therapy. This and other objects and advantages, as well as inventive features, will be apparent from the detailed description provided herein.

### SUMMARY

Provided is a pharmaceutical combination comprising (i) a chlorin-vitamin conjugate or its pharmaceutically acceptable salt, hydrate, or metal complex and (ii) at least one chemotherapeutic agent, wherein the chlorin-vitamin conjugate or its pharmaceutically acceptable salt, hydrate, or metal complex is a photosensitizer.

In some embodiments, the chlorin-vitamin conjugate can be a chlorin lipoic acid conjugate (CLA), a chlorin biotin conjugate (CBTN), a chlorin pantothenic acid conjugate (CPA), a chlorin bexarotene conjugate (CBX), a chlorin desthiobiotin conjugate (CDBTN), a chlorin biocytin conjugate (CBC), or a combination of two or more thereof.

The metal complex of the chlorin-vitamin conjugate can be formed by the chlorin moiety and a metal ion. The metal can be selected from the group consisting of zinc (Zn), indium (In), palladium (Pd), and platinum (Pt). In some embodiments, the metal is indium or zinc.

The chemotherapeutic agent used can be selected from the group consisting of paclitaxel, docetaxel, doxorubicin, cisplatin, fluorouracil, methotrexate, paraplatin, cyclophosphamide, epirubicin, gemcitabine, eribulin, ixabepilone, mutamycin, vinorelbine, capecitabine, daunorubicin, idarubicin, mitoxantrone, vinblastine, vincristine, vinorelbine, actinomycin D, bleomycin, daunomycin, and a combination of two or more thereof.

In some embodiments, the chemotherapeutic agent can be paclitaxel, doxorubicin, cisplatin, fluorouracil, methotrexate, or a combination of two or more thereof.

Provided is a pharmaceutical composition comprising therapeutically effective amounts of (i) and (ii) of the pharmaceutical combination and a pharmaceutically acceptable carrier, diluent, or excipient. The pharmaceutical composition can be comprised of nanoparticles.

Provided is a combination of pharmaceutical compositions comprising (i) a pharmaceutical composition comprising a therapeutically effective amount of the chlorin-vitamin conjugate or its pharmaceutically acceptable salt, hydrate, or metal complex and (ii) a pharmaceutical composition comprising a therapeutically effective amount of the at least one chemotherapeutic agent, wherein (i) and (ii) are independently formulated to be administered by the same or different routes. The pharmaceutical compositions either one or both of (i) and (ii) can be comprised of nanoparticles.

In some embodiments, the combination of pharmaceutical compositions (i) and (ii) can be formulated independently and can be administered intravenously, topically, or orally.

Further provided is a method for treating a patient for cancer, which method comprises administering to the patient (i) a therapeutically effective amount of a chlorin-vitamin conjugate or its pharmaceutically acceptable salt, hydrate, or metal complex and (ii) a therapeutically effective amount of at least one chemotherapeutic agent, wherein the chlorin-vitamin conjugate or its pharmaceutically acceptable salt, hydrate, or metal complex is a photosensitizer, whereupon the patient is treated for cancer.

In some embodiments, (i) and (ii) can be formulated as a single pharmaceutical composition or as separate pharmaceutical compositions, which can be administered simultaneously or sequentially by the same or different routes. In some embodiments, (i) and (ii) can be administered intravenously, topically, or orally. The therapeutically effective amount of (i) that can be administered ranges from about 30 nM to about 100 nM (such as 30 nM to 100 nM). The therapeutically effective amount of (ii) that can be administered ranges from about 25 nM to about 25 μM (such as 25 nM to 25 μM).

In some embodiments, the cancer is selected from the group consisting of head and neck cancer, breast cancer, prostate cancer, lung cancer, liver cancer, gynecological cancer, cervical cancer, brain cancer, melanoma, colorectal cancer, bladder cancer, ovarian cancer, and gastrointestinal cancer. In some embodiments, the cancer is breast cancer. In some embodiments, the breast cancer is triple-negative.

In some embodiments, (i) and (ii) of the pharmaceutical combination or pharmaceutical composition can have a synergistic effect.

### BRIEF DESCRIPTION OF THE DRAWINGS

The present disclosure will be more readily understood from the detailed description of embodiments presented below considered in conjunction with the attached drawings of which:

FIG. 1 shows the dark cytotoxicity and light treatment (cell survival assay) of triple-negative breast cancer (TNBC) cells treated with photosensitizers (PS) at 500 nM in the dark and at 100 nM in the presence of light. PSs are chlorin biotin conjugate (CBTN), chlorin bexarotene conjugate (CBX), chlorin pantothenic acid conjugate (CPA), chlorin lipoic acid conjugate (CLA), chlorin desthiobiotin conjugate (CDBTN) and their indium (In) complexes including the starting precursor methyl pheophorbide (MePheo).

FIG. 2 shows the cell survival assay of TNBC cells treated with photosensitizers (MePheo, CBTN, CPA, or InCLA) at varying concentrations of 10-100 nM, followed by light exposure.

FIG. 3 shows the cell survival assay of TNBC cells treated with Taxol® (paclitaxel) at varying concentrations of 10-100 nM followed by light exposure.

FIG. 4A shows the cell survival assay of TNBC cells treated with photosensitizers at 100 nM and at 100 nM co-treated with 50 nM paclitaxel in the dark and compared to paclitaxel-only treated cells.

FIG. 4B shows the cell survival assay of TNBC cells treated with photosensitizers at 100 nM and at 100 nM co-treated with 50 nM paclitaxel in the presence of light and compared to paclitaxel-only treated cells.

FIG. 5 shows the cell survival assay of TNBC cells treated with photosensitizers such as MePheo, CBTN, CPA, and InCLA at varying concentrations of 10-100 nM and co-treated with 50 nM paclitaxel in the presence of light, and compared to paclitaxel-only treated cells.

FIG. 6 shows the cell survival assay of TNBC cells treated with doxorubicin at varying concentrations of 100-1000 nM in the dark or in the presence of light.

FIG. 7A shows the cell survival assay of TNBC cells treated with photosensitizers at 100 nM and at 100 nM co-treated with 500 nM doxorubicin in the dark, and compared to doxorubicin-only treated cells.

FIG. 7B shows the cell survival assay of TNBC cells treated with photosensitizers at 100 nM and at 100 nM co-treated with 500 nM doxorubicin in the presence of light, and compared to doxorubicin-only treated cells.

FIG. 8 shows the cell survival assay of TNBC cells treated with photosensitizers such as MePheo, CBTN, CPA, and InCLA at varying concentrations of 10-100 nM and co-treated with 500 nM doxorubicin in the presence of light, and compared to doxorubicin-only treated cells.

FIG. 9 shows the cell survival assay of TNBC cells treated with cisplatin at varying concentrations of 4-75 μM in the dark or in the presence of light.

FIG. 10A shows the cell survival assay of TNBC cells treated with photosensitizers at 100 nM and at 100 nM co-treated with 25 μM cisplatin in the dark and compared to cisplatin-only treated cells.

FIG. 10B shows the cell survival assay of TNBC cells treated with photosensitizers at 100 nM and at 100 nM co-treated with 25 μM cisplatin in the presence of light and compared to cisplatin-only treated cells.

FIG. 11 shows the cell survival assay of TNBC cells treated with photosensitizers such as MePheo, CBTN, CPA, and InCLA at varying concentrations of 10-100 nM and co-treated with 25 μM cisplatin in the presence of light, and compared to cisplatin-only treated cells.

FIG. 12 shows the cell survival assay of TNBC cells treated with fluorouracil at varying concentrations of 10-100 μM in the dark or in the presence of light.

FIG. 13A shows the cell survival assay of TNBC cells treated with photosensitizers at 100 nM and at 100 nM co-treated with 25 μM fluorouracil in the dark, and compared to fluorouracil-only treated cells.

FIG. 13B shows cell survival assay of TNBC cells treated with photosensitizers at 100 nM and at 100 nM co-treated with 25 μM fluorouracil in the presence of light, and compared to fluorouracil-only treated cells.

FIG. 14 shows the cell survival assay of TNBC cells treated with photosensitizers such as MePheo, CBTN, CPA, and InCLA at varying concentrations of 10-100 nM and co-treated with 25 μM fluorouracil in the presence of light, and compared to fluorouracil-only treated cells.

FIG. 15 shows the cell survival assay of TNBC cells treated with methotrexate at varying concentrations of 10-100 nM in the dark or in the presence of light.

FIG. 16A shows the cell survival assay of TNBC cells treated with photosensitizers at 100 nM and at 100 nM co-treated with 500 nM methotrexate in the dark, and compared to methotrexate-only treated cells.

FIG. 16B shows the cell survival assay of TNBC cells treated with photosensitizers at 100 nM and at 100 nM co-treated with 500 nM methotrexate in the presence of light, and compared to methotrexate-only treated cells.

FIG. 17 shows the cell survival assay of TNBC cells treated with photosensitizers such as MePheo, CBTN, CPA, and InCLA at varying concentrations of 10-100 nM and co-treated with 500 nM methotrexate in the presence of light, and compared to methotrexate-only treated cells.

FIG. 18 shows fluorescence microscopy images of fixed human TNBC BT-549 cells. Grayscale images of cells stained with Hoechst 33258. Morphology of (A) untreated unirradiated cells, sham control; (B) illuminated cells; (C-E) cells treated with: PSs (InCLA, CBTN, and CPA) and paclitaxel in the dark; (F-H) PSs, followed by 1 minute light exposure; (I) paclitaxel unirradiated; (J-L) PSs and paclitaxel in the dark; (M-O) PSs co-treated with paclitaxel, then light-exposed; (P-Q) PSs co-treated with doxorubicin, then irradiated; (R) PSs and cisplatin, dark; (S-T) PSs and cisplatin, irradiated; (U) fluorouracil, dark; (V) InCLA with fluorouracil in the dark; (W) light-exposed and, (X-Y) PSs with methotrexate with light. Inserts indicate enlarged view. Shrunk cells and chromatin condensation are evident upon treatment with paclitaxel (I), on irradiated cells co-treated with PSs and paclitaxel (M-O), and with cisplatin (S-T). Light dose in 1 minute=0.96 J cm⁻²; in 30 seconds=0.48 J cm⁻². Concentrations: PSs=50 nM; paclitaxel=50 nM; doxorubicin=500 nM; cisplatin=25 μM; fluororuracil=25 μM; and, methotrexate=500 nM.

FIG. 19. shows ultrastructure TEM images of human triple-negative breast cancer (TNBC) BT-549 cells: (A) untreated irradiated; (B) PS-treated with InCLA and irradiated; (C) paclitaxel-treated in the dark; (D) co-treatment with InCLA and paclitaxel unirradiated; (E) paclitaxel-treated only and irradiated; (F) co-treatment with InCLA and paclitaxel, followed by light treatment; (G) co-treatment with InCLA and FU (fluorouracil) in the dark, and (H) co-treatment with InCLA and FU with light treatment. Irregularly shaped nuclear membrane is apparent in treated cells in the dark or in the presence of light. Light dose in 30 seconds=0.48 J cm⁻². Concentrations: PSs=50 nM; paclitaxel=50 nM; and, fluororuracil=25 μM.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the claimed invention is thereby intended.

The term “photodynamic therapy (PDT)” or “light treatment” refers to an anti-cancer therapy that uses a photosensitizer (PS) which, upon light activation, produces cytotoxic oxygen species destroying tumor cells.

The term “photosensitizer (PS)” refers to a drug when absorbed by cancer cells and exposed to light, the drug becomes active and kills the cancer cells.

The term “combination therapy” refers to therapy that combines more than one method of treatment or more than one drug to treat a disease.

The terms “chemotherapeutic agent”, “anti-cancer drug”, and “anti-cancer prodrug” refers to drugs (i.e., chemical compounds) or prodrugs known to, or suspected of being able to treat cancer (i.e., to kill cancer cells, prohibit proliferation of cancer cells, or treat a symptom related to cancer). In some embodiments, the term “chemotherapeutic agent” refers to a non-PS molecule that is used to treat cancer and/or that has cytotoxic ability.

The drug combination is the most widely used technique to treat dreadful diseases such as cancer. The main purpose of the combination is to achieve a synergistic therapeutic effect, dose, and toxicity reduction, thereby diminishing or delaying the induction of drug resistance. Synergism can cause minimization of toxicity effects and drug resistance. Photodynamic therapy (PDT) or light treatment has the ability to destroy tumors sparing normal tissue structures surrounding the tumor, whereas chemotherapy is a well-established standard treatment provided to cancer patients using chemotherapeutic agents to destroy cancer cells. However, these therapies have their side effects, such as safety issues and multi-drug resistance. Combination therapies can overcome efficacy limitations while lessening the treatment-associated morbidity of both therapies.

In view of the above, the present disclosure provides a combination chemophotodynamic (chemoPDT) therapy that can include PDT and chemotherapy (chemo) by using a photosensitizer (PS) compound and at least one chemotherapeutic agent in combination for treating various types of cancer.

Provided is a pharmaceutical combination comprising (i) a chlorin-vitamin conjugate or its pharmaceutically acceptable salt, hydrate, or metal complex and (ii) at least one chemotherapeutic agent, wherein the chlorin-vitamin conjugate or its pharmaceutically acceptable salt, hydrate, or metal complex is a PS.

The chlorin-vitamin conjugates can be used as PSs for PDT. The chlorin-vitamin conjugates and the method of their preparation are disclosed in U.S. Pat. Nos. 10,806,788 and 11,191,835, which are hereby specifically incorporated by reference for their teachings regarding the same. Chlorin-vitamin conjugates provide low dark toxicity and high selectivity for tumors over normal tissues.

The chlorin-vitamin conjugate is composed of a chlorin moiety such as methyl pheophorbide (MePheo, a chlorin derivative) conjugated to a vitamin moiety optionally with their corresponding metal complex. Chlorin moiety can be light-activable and derived from plant-based chlorophyll.

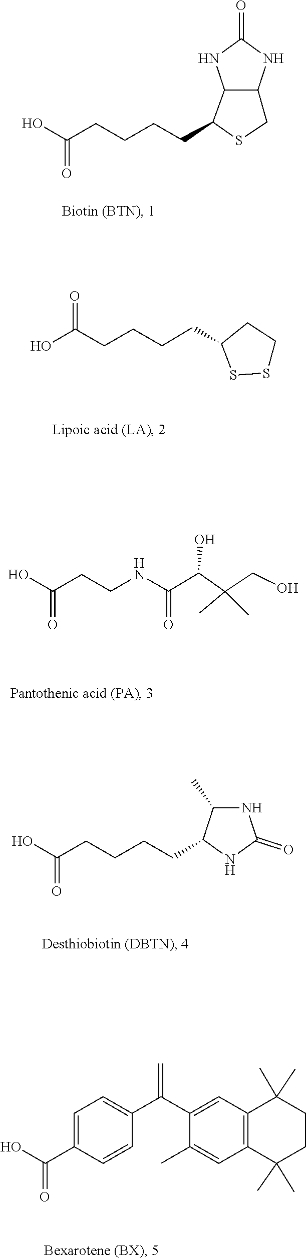 [SMILES: O=C(O)CCCC[C@@H]1SCC2NC(=O)NC21 ; O=C(O)CCCC[C@@H]1CCSS1 ; CC(C)(CO)[C@@H](O)C(=O)NCCC(=O)O ; C[C@@H]1NC(=O)N[C@@H]1CCCCC(=O)O ; C=C(c1ccc(C(=O)O)cc1)c1cc2c(cc1C)C(C)(C)CCC2(C)C]

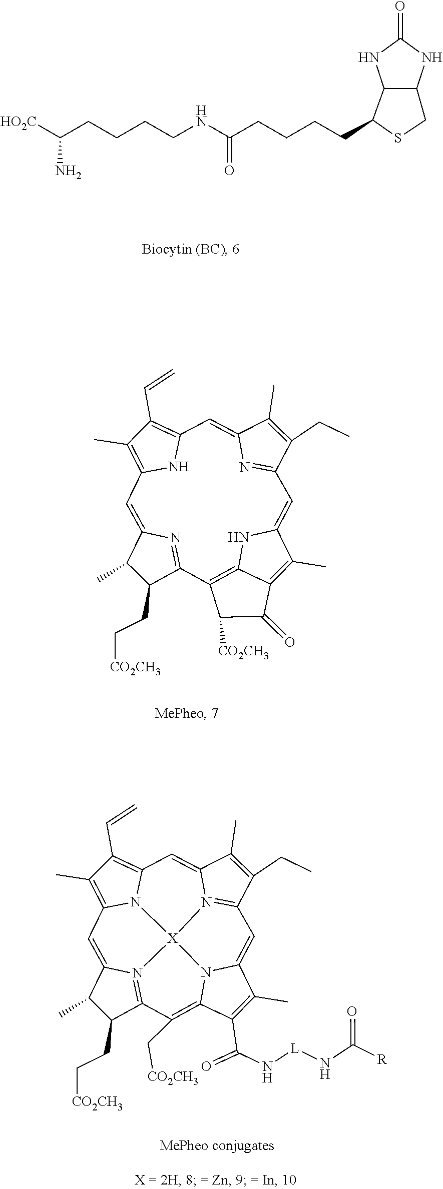

Molecular structures of vitamins and vitamin analogues wherein, R is a carbon chain of vitamin or its analogue without CO₂H from biotin (BTN), bexarotene (BX), lipoic acid (LA), pantothenic acid (PA), desthiobioitn (DBTN), and biocytin (BC); L is —(CH₂)₆—; and X is H, Zn or In.

Vitamin moiety is selected from the group consisting of biotin (Vit H or B7), bexarotene (Vit A analogue), lipoic acid (coenzyme), pantothenic acid (Vit B5), desthiobiotin (biotin analogue), and biocytin (biotin metabolite).

In some embodiments, the chlorin-vitamin conjugate is a chlorin lipoic acid conjugate (CLA), a chlorin biotin conjugate (CBTN), a chlorin pantothenic acid conjugate (CPA), a chlorin bexarotene conjugate (CBX), a chlorin desthiobiotin conjugate (CDBTN) or a chlorin biocytin conjugate (CBC) or a combination two or more thereof.

Chlorin-vitamin conjugate is a metal complex formed by the chlorin moiety and a metal ion. The metal is selected from the group consisting of zinc (Zn), indium (In), palladium (Pd), and platinum (Pt). In some embodiments, the metal is indium or zinc.

Chlorin-vitamin conjugates were prepared by linking a chlorin (chlorophyll derivative) such as MePheo (7) and a vitamin or vitamin analogue to create MePheo-conjugates (8) and their Zn and In complexes (9-10).

The chemotherapeutic agent can be used to provide chemotherapy in combination with PDT. The chemotherapeutic agent is selected from the group consisting of paclitaxel, docetaxel, doxorubicin, cisplatin, fluorouracil, methotrexate, paraplatin, cyclophosphamide, epirubicin, gemcitabine, eribulin, ixabepilone, mutamycin, vinorelbine, capecitabine, daunorubicin, idarubicin, mitoxantrone, vinblastine, vincristine, vinorelbine, actinomycin D, bleomycin, daunomycin, and a combination of two or more thereof.

In some embodiments, the chemotherapeutic agent is paclitaxel, doxorubicin, cisplatin, fluorouracil, methotrexate, or a combination of two or more thereof. The structures of the chemotherapeutic agents are:

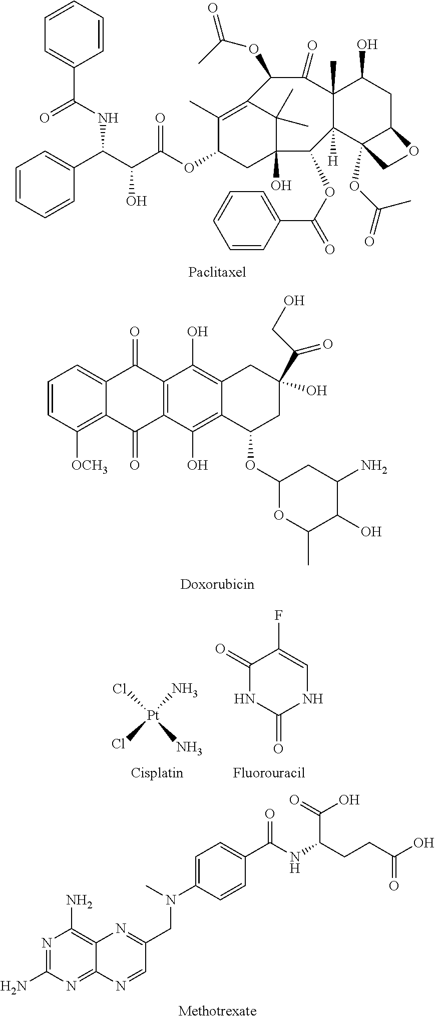

These chemotherapeutic agents are selected based on their action of mechanism.

Paclitaxel is a chemotherapy medication used to treat a variety of tumors (breast, lungs, pancreatic, ovarian, oesophageal, cervical, Kaposi's sarcoma) administered intravenously or as an albumin-bound formulation. The mechanism of therapeutic action of paclitaxel is based on its ability to bind to microtubules and promote the assembly of alpha and beta tubulin subunits, which are the building blocks of microtubules. Paclitaxel interferes with the dynamics of microtubule polymerization, delays the progression of mitosis by inducing failure in chromosomal segregation, eventually leading to induction of apoptosis and mitotic arrest, and eventually tumor destruction.

Doxorubicin, an anthracycline antibiotic, is the treatment of choice as first-line chemotherapy for metastatic breast cancer patients not previously treated with anthracyclines. One of the mechanisms proposed for the anti-cancer activity of doxorubicin is the intercalation into the DNA as other anthracyclines leading to disruption of DNA repair. It inhibits topoisomerase II supercoiling of the DNA during transcription, preventing DNA recombination of the double helix and blocking DNA replication.

Cisplatin binds to the reactive center on purine residues and can cause DNA damage in cancer cells, blocking cell division and resulting in apoptotic cell death. The 1,2-intrastrand cross-links of purine bases with cisplatin are the most notable among the changes in DNA. These molecular mechanisms of cytotoxicity constitute the hallmarks of cisplatin bioactivity. Sporadic triple-negative breast cancer displays aberrant DNA repair and genomic instability, stipulating the rationale for using platinum-based entities that provoke DNA damage.

5-Fluorouracil (5FU) is the first rationally designed antimetabolite chemotherapeutic drug widely used either alone or in combination with other drugs to treat solid tumors of digestive origins (gastric, oesophageal, colorectal, anal, and pancreatic) and those arising in other organs (breast, cervix, and head and neck). The synthetic design of 5FU was based on the observation that tumor tissues utilize uracil more rapidly than normal tissues. The anti-folate or anti-vitamin folic acid property of fluoropyrimidines is thought to be the principal mechanism of action. The key enzyme inhibited by 5FU is thymidylate synthase (TS), which is responsible for the de novo synthesis of dTMP. Its uniqueness to target TS expression independent of breast cancer status and its cost-effectiveness compared to other chemotherapeutic drugs make 5FU an attractive choice for breast cancer treatment.

Cellular absorption of methotrexate uses folate receptors via receptor-mediated endocytosis, which is responsible for internalizing bound folates or folate conjugates. Methotrexate is commonly utilized for the treatment of various neoplastic diseases (breast, bladder, leukemia, osteosarcoma, and a number of other cancers).

In some embodiments, the pharmaceutical combination used in the combination therapy can have a synergestic effect at a nanomolar concentration of PSs and chemeotherapeutic agents.

Provided is a pharmaceutical composition comprising therapeutically effective amounts of (i) and (ii) of the pharmaceutical combination and one or more pharmaceutically acceptable carrier, diluent, or excipient.

The pharmaceutical composition can be administered by any suitable route. In some embodiments, the suitable route can be intravenous, topical, or oral.

Further, provided is a combination of pharmaceutical compositions comprising (i) a pharmaceutical composition comprising a therapeutically effective amount of the chlorin-vitamin conjugate or its pharmaceutically acceptable salt, hydrate, or metal complex, and (ii) a pharmaceutical composition comprising a therapeutically effective amount of the at least one chemotherapeutic agent, wherein (i) and (ii) are independently formulated to be administered by the same or different routes.

The combination of pharmaceutical compositions can be formulated independently and administered intravenously, topically, or orally.

In the combination of pharmaceutical compositions, either one or both of (i) and (ii) can comprised of nanoparticles.

Further, provided is a method for treating a patient for cancer, which method comprises: administering to the patient (i) a therapeutically effective amount of a chlorin-vitamin conjugate or its pharmaceutically acceptable salt, hydrate, or metal complex and (ii) a therapeutically effective amount of at least one chemotherapeutic agent, wherein the chlorin-vitamin conjugate or its pharmaceutically acceptable salt, hydrate, or metal complex is PS, whereupon the patient is treated for cancer.

Examples of cancer include, but are not limited to, head and neck cancer, breast cancer, prostate cancer, lung cancer, liver cancer, gynecological cancer, cervical cancer, brain cancer, melanoma, colorectal cancer, bladder cancer, ovarian cancer, and gastrointestinal cancer.

In some embodiments, the cancer is breast cancer. Examples of breast cancer include, but are not limited to, triple-negative breast cancer (TNBC) or triple-positive breast cancer (TPBC). In some embodiments, the breast cancer is triple-negative breast cancer (TNBC).

In some embodiments, (i) the chlorin-vitamin conjugate, or its pharmaceutically acceptable salt, hydrate, or metal complex and (ii) at least one chemotherapeutic agent, can be formulated as a single pharmaceutical composition or as separate pharmaceutical compositions, which can be administered simultaneously or sequentially, in either order by the same or different routes. In some embodiments, (i) and (ii) can be administered intravenously, topically, or orally.

The dosage levels of compounds used in the compositions described herein can be varied to administer an amount of the composition that is effective in achieving the desired effect for a particular patient. The selected dosage level can depend upon the activity of the composition and the route of administration.

The chlorin vitamin conjugates can be administered in the therapeutically effective amount from about 10 nM to about 100 nM, such as about 10 nM to 100 nM, 10 nM to about 100 nM, or 10 nM to 100 nM. Desirably, the therapeutically effective amount is from about 30 nM to about 60 nM, such as about 30 nM to 60 nM, 30 nM to about 60 nM, or 30 nM to 60 nM.

The method of treatment can further comprise subjecting the patient to light treatment. The light dose used can correspond to an energy fluence rate of about 0.96 J/cm² (such as 0.96 J/cm²) and a power of about 16 mW/cm² (such as 16 mW/cm²).

The chemotherapeutic agents can be administered in the therapeutically effective amount from about 5 nM to about 50 μM, such as about 5 nM to 50 μM, 5 nM to about 50 μM, or 5 nM to 50 μM. In some embodiments, paclitaxel, doxorubicin, and methotrexate can be administered in the therapeutically effective amount from about 30 nM to about 1,000 nM, such as about 30 nM to 1,000 nM, 30 nM to about 1,000 nM, or 30 nM to 1,000 nM. Desirably, the therapeutically effective amount is about 50 nM to about 500 nM, such as about 50 nM to 500 nM, 50 nM to about 500 nM, or 50 nM to 500 nM. In some embodiments, cisplatin and fluorouracil can be administered in the therapeutically effective amount from about 0 μM to 50 μM, such as about 0 μM to 50 μM, 0 μM to about 50 μM, or 0 μM to 50 μM. Desirably, the therapeutically effective amount is about 20 μM to about 40 μM such as about 20 μM to 40 μM, 20 μM to about 40 μM, or 20 μM to 40 μM.

The combination chemoPDT therapy for TNBC cells can consist of about 24 hour treatment with a chlorin-vitamin conjugate and a chemotherapeutic agent. The co-treated cells can be subjected to light treatment. The combination therapy can show synergistic enhancement of the anti-cancer effect. The synergistic effect can be achieved at the nanomolar concentration of each compound. Thus, the combination therapy can reduce the dosage of each chlorin-vitamin conjugate and chemotherapeutic agent compared to their monotherapy which may decrease the overall side effects.

TABLE 1
shows cell inhibition (%) of TNBC cells treated with photosensitizers
(100 nM) and chemotherapeutic drugs for 24 h, followed by light
irradiation (light dose of 0.96 J cm −2). Chemotherapeutic
drugs: paclitaxel (50 nM), doxorubicin (500 nM), cisplatin (25
μM), fluorouracil (25 μM); and, methotrexate (500 nM).

| Photosen- | Pacli- | | | | |
|---|---|---|---|---|---|
| sitizer | taxel | Doxorubicin | Cisplatin | Fluorouracil | Methotrexate |
| MePheo | 55 | 36 | 39 | 34 | 40 |
| CBTN | 65 | 64 | 53 | 64 | 42 |
| CBX | 48 | 30 | 0 | 6 | 42 |
| CPA | 63 | 73 | 87 | 43 | 33 |
| CLA | 45 | 33 | 65 | 35 | 43 |
| InCBTN | 45 | 25 | 0 | 13 | 33 |
| InCBX | 45 | 41 | 16 | 9 | 42 |
| InCPA | 51 | 27 | 0 | 25 | 30 |
| InCLA | 91 | 91 | 90 | 92 | 61 |

Based on these data of the PSs at 100 nM concentration, InCLA exhibited the most potency either alone or in combination, specifically with paclitaxel, doxorubicin, cisplatin, and fluorouracil. Following InCLA were CPA and CBTN having the best chemophotodynamic efficacy among the synthesized PSs tested. InCLA, CPA, and CBTN showed better activity against TNBC cells than the starting compound MePheo. The best combination for CBTN is with paclitaxel, followed by doxorubicin or fluorouracil, while for CPA, cisplatin is the best, next doxorubicin, then paclitaxel.

TABLE 2
shows combination index (CI) values of binary therapy
(selected PSs and chemotherapeutic drugs) and the corresponding
synergism vs additive vs antagonism effects.

| | Pacli- | | | | |
|---|---|---|---|---|---|
| PSs | taxel | Doxorubicin | Cisplatin | Fluorouracil | Methotrexate |
| 50 nM | 50 nM | 500 nM | 25 μM | 25 μM | 500 μM |
| MePheo | 13.27 | 6.37 | 6.10 | 17.96 | 7.79 |
| | (VSA) | (SA) | (SA) | (VSA) | (SA) |
| CBTN | 0.74 | 2.44 | 0.99 | 0.48 | 1.57 |
| | (MS) | (A) | (NA) | (S) | (A) |
| CPA | 0.41 | 1.52 | 1.30 | 0.45 | 0.50 |
| | (S) | (A) | (MA) | (S) | (S) |
| InCLA | 0.25 | 1.91 | 1.09 | 0.94 | 1.21 |
| | (SS) | (MA) | (NA) | (NA) | (MA) |

CI = 1 (additive); CI < 1 (synergism); CI > 1 (antagonism).
VSS (<0.1), very strong synergism; SS (0.1-0.3), strong synergism; S (0.3-0.7), synergism; MS (0.7-0.85), moderate synergism; StS (0.85-0.90), slight synergism; NA (0.9-1.10), nearly additive; StA (1.10-1.20), slight antagonism; MA (1.20-1.45), moderate antagonism; A (1.45-3.3), antagonism; SA (3.3-10), strong antagonism; VSA (>10), very strong antagonism.
Concentrations: PSs = 50 nM; paclitaxel = 50 nM; doxorubicin = 500 nM; cisplatin = 25 μM; fluorouracil = 25 μM; methotrexate = 500 nM.

The CI values for PSs selected based on the best biological activity to inhibit the growth of TNBC cells in combination with the chemotherapeutic agents, listed in Table 2 as calculated using CompuSyn software. The PSs showed better results than the starting compound MePheo and confirmed the results in the cell viability assay. The combination of InCLA and paclitaxel showed the strongest synergism with the lowest CI value of 0.25. Other combinations in the table range from synergistic to nearly additive, with CI values ranging from 0.41-1.09. The calculated CI values are based on 50 nM concentration for the PSs, 50 nM paclitaxel, 500 nM doxorubicin, 25 μM cisplatin, 25 μM fluorouracil, and 500 nM methotrexate.

In some embodiments, the chlorin vitamin conjugate and chemotherapeutic agent can be used in a ratio of about 1:1.5 to 1:500 (such as 1:1.5 to 1:500).

The method comprises administering a therapeutically effective amount of chlorin vitamin conjugate and at least one chemotherapeutic agent as neat compounds or as a pharmaceutical composition. The compounds or a pharmaceutical composition can be administered during or after the onset of the disease or condition.

Further, provided is a method for treating cancer in a patient, which method comprises: administering to a patient a therapeutically effective amount of a pharmaceutical combination or a pharmaceutical composition described herein and one or more therapeutic agents.

The methods of combination therapy disclosed herein can result in a synergistic effect, wherein the effect of a combination of compounds or other therapeutic agents is greater than the sum of the effects resulting from the administration of any of the compounds or other therapeutic agents as single agents. A synergistic effect may also be an effect that cannot be achieved by administration of any of the compounds or other therapeutic agents as single agents. The synergistic effect may include, but is not limited to, an effect of treating cancer by reducing tumor size, inhibiting tumor growth, or increasing survival of the subject. The synergistic effect may also include reducing cancer cell viability, inducing cancer cell death, and inhibiting or delaying cancer cell growth.

The term “therapeutically effective amount” or “therapeutically effective dose” refers to an amount of the active ingredient(s) that is (are) sufficient, when administered, to deliver efficaciously the active ingredient(s) for the treatment of a disease or condition of interest to a patient in need thereof. The prophylactically or therapeutically effective amount of such combination will vary depending upon the patient and the disease or condition being treated, the weight and age of the patient, the severity of the disease or condition, the manner of administration, and the like, which can readily be determined by one of ordinary skill in the art. In the case of a cancer or other proliferative disorder, the prophylactically or therapeutically effective amount of the agent may reduce (i.e., inhibit to some extent or stop) unwanted cellular proliferation; reduce the number of cancer cells; reduce the tumor size; inhibit (or stop) cancer cell infiltration into peripheral organs; inhibit (or stop) tumor metastasis; inhibit, e.g., to some extent, tumor growth; and/or relieve, to some extent, one or more of the signs or symptoms associated with the cancer. To the extent the administered compound or composition prevents growth and/or kills existing cancer cells, it may be cytostatic and/or cytotoxic.

For any compound, a therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. The term “treat” and synonyms contemplate administering a prophylactic or therapeutically effective amount of a combination or composition described herein to a subject in need of such treatment. The treatment can be orientated symptomatically, for example, to suppress symptoms. It can be effected over a short period, be oriented over a medium term, or can be a long-term treatment, for example within the context of maintenance therapy.

The term “pharmaceutical composition” includes a therapeutically effective amount of one or more compounds for treating a cancer patient. The composition may include other components and/or ingredients, including, but not limited to, other therapeutically active compounds and/or one or more pharmaceutically acceptable carriers, diluents, excipients, and the like. The carrier, excipient, or diluent can vary based on the particular route of administration (see, e.g., Remington's The Science and Practice of Pharmacy, 23^{rd} ed. (2020)).

Generally, daily oral doses of a compound are from about 0.01 milligrams/kg per day to 1,000 milligrams/kg per day. Oral doses in the range of 0.5 to 50 milligrams/kg, in one or more administrations per day, can yield therapeutic results. Dosage can be adjusted appropriately to achieve desired drug level, local or systemic, depending upon the mode of administration. For example, intravenous administration can vary from one order to several orders of magnitude lower dose per day. If the response in a subject is insufficient at such doses, even higher doses (or effective higher doses by a different, more localized delivery route) can be employed to the extent that patient tolerance permits. Multiple doses per day are contemplated to achieve appropriate systemic levels of the compound.

The compounds can be typically administered in admixture with a pharmaceutical carrier to give a pharmaceutical composition selected with regard to the intended route of administration and standard pharmaceutical practice. Pharmaceutical compositions can be formulated in a conventional manner using one or more physiologically acceptable carriers comprising excipients and/or auxiliaries that facilitate the processing of the compound. The pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of a compound described herein is administered orally, the composition typically is in the form of a tablet, capsule, powder, solution, or elixir. When administered in tablet form, the composition additionally can contain a solid carrier, such as a gelatin or an adjuvant. The tablet, capsule, and powder can contain about 0.01% to about 95%, and preferably from about 1% to about 50%, of the combination of compounds. When administered in liquid form, a liquid carrier can be added, such as water, petroleum, or oils of animal or plant origin. The liquid form of the composition can further contain the physiological saline solution, dextrose or other saccharide solutions, or glycols. When administered in liquid form, the composition contains about 0.1% to about 90%, and preferably about 1% to about 50%, by weight, of the combination of compounds.

For oral administration, the compounds can be formulated readily by combining the active compound(s) with pharmaceutically acceptable carriers, excipients, or diluents well-known in the art. Such carriers, excipients, or diluents enable the compounds to be formulated as tablets, pills, powders, dragees, capsules, liquids, gels, syrups, slurries, suspensions, solutions, and the like for oral ingestion by a subject to be treated.

The exact formulation, route of administration, and dosage of a pharmaceutical composition comprising an effective amount of the compound are determined by an individual physician in view of the diagnosed condition or disease. The dosage amount and interval can be adjusted individually to provide levels of the compound that are sufficient to maintain a prophylactic or therapeutic effect.

Toxicity and therapeutic efficacy of the combination can be determined by standard pharmaceutical procedures in cell cultures or experimental animals, e.g., for determining the maximum tolerated dose (MTD) of a compound, which is defined as the highest dose that causes no toxicity in animals. The therapeutic index is the dose ratio between the maximum tolerated dose and therapeutic effects (e.g., inhibition of tumor growth). The dosage can vary within this range depending upon the dosage form employed and the route of administration utilized. The determination of a therapeutically effective amount is well within the capability of those ordinarily skilled in the art, especially in light of the detailed disclosure provided herein.

A combination can be administered in an amount of about 0.005 to about 500 milligrams per dose, about 0.05 to about 250 milligrams per dose, or about 0.5 to about 100 milligrams per dose. For example, the combination can be administered, per dose, in an amount of about 0.005, about 0.05, about 0.5, about 5, about 10, about 20, about 30, about 40, about 50, about 100, about 150, about 200, about 250, about 300, about 350, about 400, about 450, or about 500 milligrams, including all doses between 0.005 and 500 milligrams.

As stated above, a combination or composition described herein can be administered in with one or more other prophylactically or therapeutically active agents.

It will be appreciated by persons skilled in the art that the present disclosure is not limited by what has been particularly shown and described herein above. Rather the scope of the present disclosure includes both combinations and sub-combinations of the various features described hereinabove as well as variations and modifications which would occur to persons skilled in the art upon reading the specification and which are not in the prior art.

### Examples

The following examples serve to illustrate the present disclosure. The examples are not intended to limit the scope of the claimed invention in any way.

Human mammary epithelial carcinoma cell lines purchased from the American Type Culture Collection BT-549 (ATCC HTB-122) were cultured according to ATCC protocol. BT-549 cells were grown in RPMI 1640 containing 0.023 IU/mL insulin and supplemented with 10% fetal bovine serum (FBS). Cells were grown to 80-90% confluence in 75-cm² culture flasks (Corning) for 4-5 days in a humidified incubator (Fisher Scientific Isotemp) with 5% CO₂ at 37° C. During the incubation period, growth media was changed once with fresh pre-warmed media (pH 7.2). To harvest the cells, old growth media was aspirated out, and 3 mL 0.25% trypsin solution (Thermo Sci Hyclone) were added. The cells were incubated for 5 minutes, and the cell pellet after centrifugation was resuspended in 3 mL media, broken up gently, then 1 mL of suspended cells was transferred into a new T75 cell culture flask containing pre-warmed media (20 mL) for further culturing.

### Cell Cytotoxicity Assay

Cells were grown to confluence in a 96-well plate (9×10³ cells/well) and co-treated for 24 hours with synthesized photosensitizers (PSs) and chemotherapeutic agents of varying concentrations ranging from 5 nM to 50 μM) in growth media from a stock solution of 10 mM in DMSO (dimethyl sulfoxide, Fisher). After 24-hour treatment, old growth media containing the PSs or compounds were aspirated out and replaced with fresh media. Plates were then positioned below a non-coherent LumaCare LC-122 650 nm light source for 1 minute at an energy fluence rate of 0.96 J/cm² and a power of 16 mW/cm² (measured using a Newport optical power meter Model 840). Unirradiated cells served as control samples. The following day, cells were washed with pre-warmed PBS, and 11MTT (3-[4,5-dimethyl-thiazol-2-yl]-2.5-diphenyltetrazolium bromide, Sigma, 0.3 mg/mL) in PBS was added to each well. Samples were allowed to incubate for additional 2 hours, after which dark blue crystals formed. DMSO was added to each well and plates were shaken at room temperature for 1 hour to dissolve the purplish-blue formazan crystals. Absorbance values at 570 nm were measured on a microplate reader (BioRad 550). Cell survival was calculated based on the absorbance of the untreated cells alone (as control) and were directly proportional to the number of viable cells in culture. Results were reported as the mean±SD of triplicate measurements.

### Fluorescence Microscopy

Cells (1 mL aliquots) obtained from a diluted cell suspension were seeded into each well (1.7 cm², 8×10³ cells/well) of a 4-well culture slide (BD Biosciences) and grown to confluence in 5% CO₂ at 37° C. for 3-4 days for attachment to the substratum. After aspirating the old growth media, 1 mL of the photosensitizer and chemotherapeutic agents (with appropriate concentrations) in fresh pre-warmed media at 37° C. was added to each well. After compound treatment for 24 hours, cells were washed twice with 1 mL fresh growth media, and then irradiated with light using LumaCare LC-122 as described above. Cells were stained in the dark with Hoechst 33258 (Molecular Probes) in pre-warmed media for 10 minutes at 37° C., washed twice with filtered PBS, then fixed with filtered paraformaldehyde for 15 minutes in the incubator. After thorough liquid aspiration, the wells were removed and allowed to air dry in the dark for 1 hour. Slides were protected with coverslips, whose edges were sealed using clear fast-drying nail polish and allowed to dry at room temperature in the dark for 30 minutes. Images were recorded using fluorescence microscopy (DAPI for Hoechst 350-390 nm excitation and 460-490 nm emission filters) using an upright fluorescence microscope with Retiga imaging 2000R (Nikon Optiphot-2, 20× and 40×) and an image processing Nikon NIS-Elements V4.0 Qimaging software.

### Transmission Electron Microscopy

Cells were cultured to confluence in a petri dish (50 cm in diameter), treated for 24 hrs with 500 nM of PS, then irradiated for 2 minutes as above. After 12 photosensitization 24 hours later, cells were scraped gently in the dark, fixed in 2.5% glutaraldehyde in 0.1M sodium cacodylate buffer, then post-fixed with 1% osmium tetroxide (containing 0.8% ferricyanide), treated with 2% aqueous uranyl acetate, and subsequently dehydrated in gradient concentrations (50-100%) of varying ethanol:water mixtures. The resulting pellets were embedded in resin and consequently cut with an ultramicrotome to a 70 nm thickness, then viewed using Tecnai T20 transmission electron microscope.

Cell Viability after Combination Treatment in Triple-Negative Breast Cancer Cells

The unmetallated PS and the indium complexes were used.

Light dose corresponds to an energy fluence rate of 0.96 J/cm² used.

Power of 16 mW/cm² used.

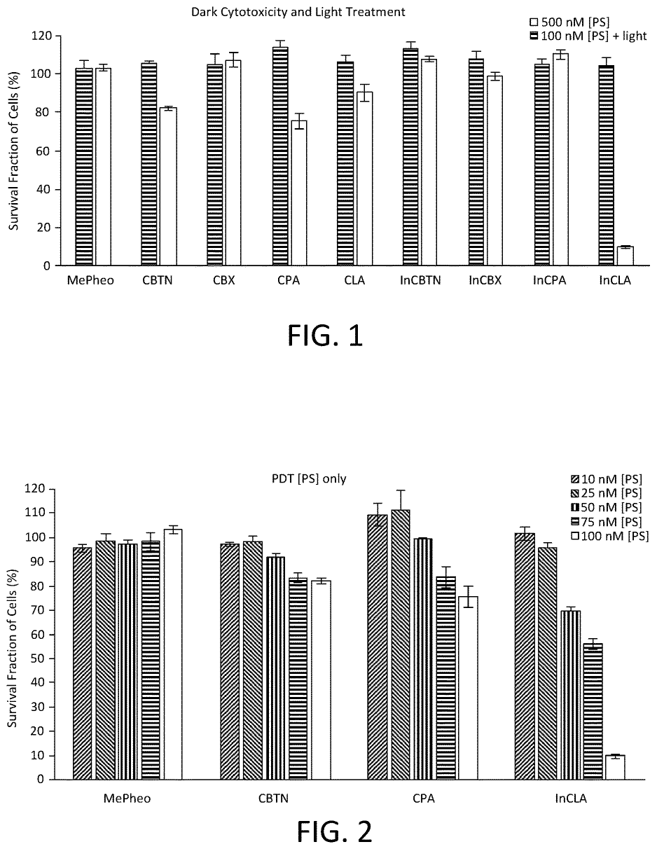

The dark cytotoxicity and light treatment of PS-treated TNBC cells are shown in FIG. 1. PSs are CBTN, CBX, CPA, CLA, and their In complexes, including the starting precursor MePheo PSs concentration was 500 nM and 100 nM in the dark and light-treated, respectively. No cytotoxicity was observed in the absence of light, and InCLA (9.6% cell survival) was the most phototoxic followed by CPA (75.3%) and CBTN (82.0%) at 100 nM. TNBC cell survival at low concentrations (10-100 nM) of selected PSs (CBTN, CPA, InCLA, and MePheo) is shown in FIG. 2 with InCLA exhibiting a significant dose-dependent response compared to the other PSs considered.

### i) PDT and Paclitaxel

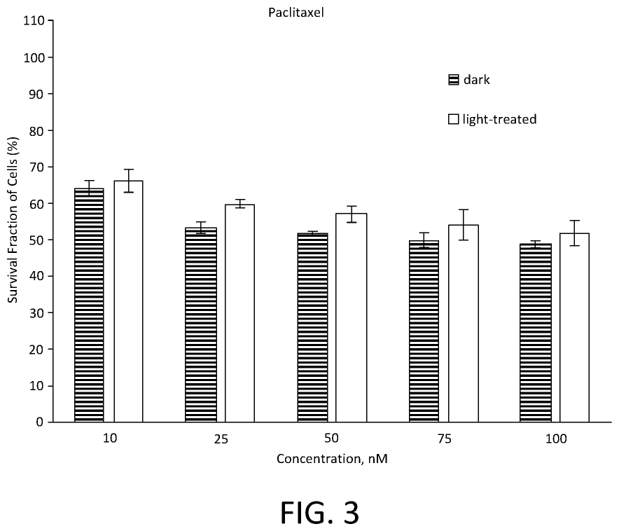

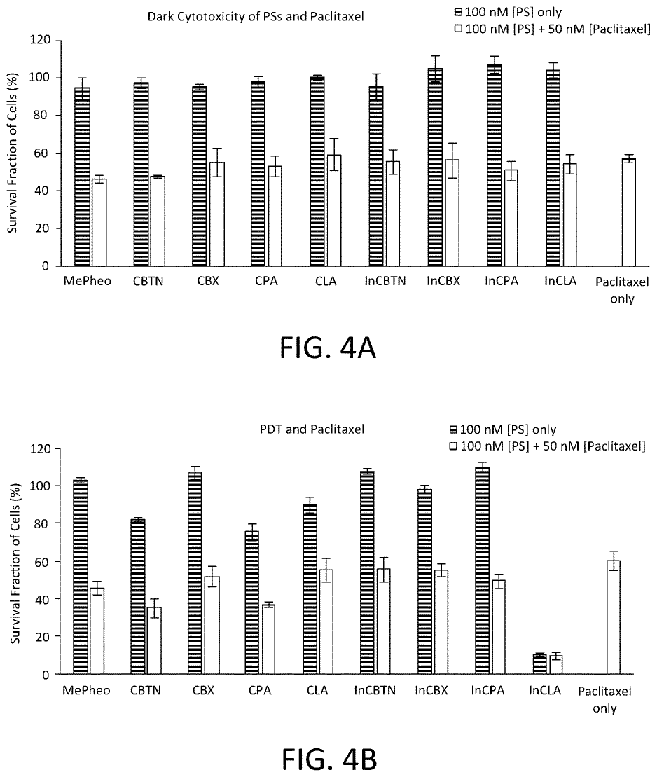

Cytotoxicity of paclitaxel at varying concentrations of 10-100 nM in TNBC cells in the dark and upon light exposure is shown in FIG. 3. Cell survival decreased from 64% to 49% and from 66% to 52% for paclitaxel-only treated cells in the dark and upon light irradiation, respectively. A very low increase (average of 4%) in cell survival was observed with the paclitaxel-treated cells upon light irradiation compared to unirradiated cells. PS-treated cells (at 100 nM) in the dark showed no cytotoxicity (FIG. 4A). Co-treatment of TNBC cells with PS (100 nM) and paclitaxel (50 nM) for 24 hours without light exposure resulted in an average of 53% cell survival (46% lowest for MePheo and 59% highest for CLA) compared to paclitaxel-only-treated cells with 57% survival. In the absence of light, only the cytotoxic effect of paclitaxel was effective.

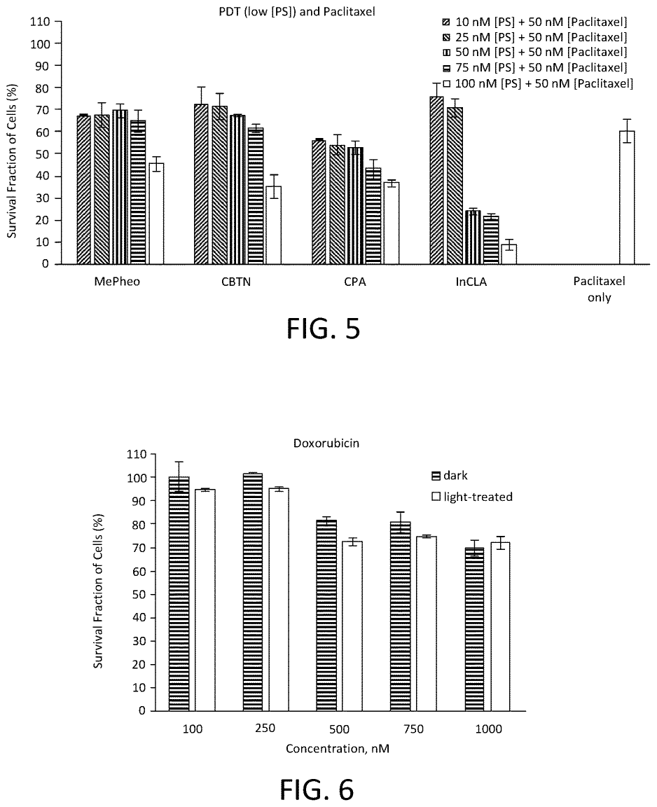

Upon irradiation with light of co-treated cells with 50 nM paclitaxel (FIG. 4B), InCLA (at 100 nM) was the most phototoxic with an observed 10% cell survival, followed by CBTN and CPA with 35% and 37% cell survival, respectively. At the concentration of 100 nM, the photodynamic effect of InCLA prevailed and overpowered the effect of paclitaxel. Except for InCLA, the average decrease in cell viability of TNBC cells was 50% upon co-treatment with 50 nM paclitaxel compared to PS-treatment alone at 100 nM. There was an average of 7% decrease between combination therapy compared to paclitaxel treatment alone (FIG. 4B). The best PSs (MePheo, CBTN, CPA, and InCLA) that responded to co-treatment with paclitaxel was tested at lower concentrations of 10-100 nM as shown in FIG. 5. At a lower concentration of 50 nM for both InCLA and paclitaxel, photodynamic cell destruction dramatically increased as indicated by the amount of cells surviving from 80% for PS-only treatment (FIG. 2) to 24% in the presence of both InCLA and paclitaxel indicating a synergistic effect in TNBC cells (FIG. 5). Combination therapy (PS and paclitaxel) was observed to be better than monotherapy alone.

### ii) PDT and Doxorubicin

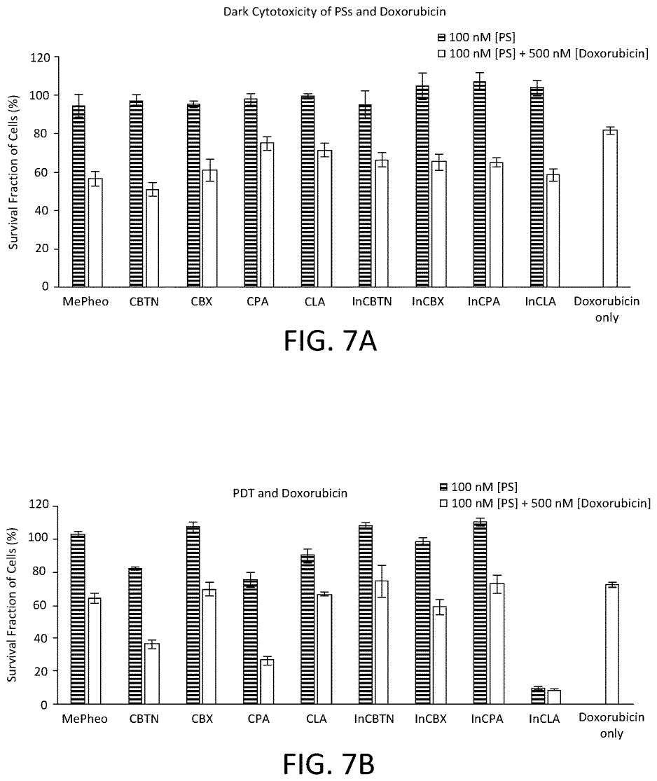

Cytotoxicity of doxorubicin at varying concentrations of 100-1000 nM in TNBC cells in the dark and upon light exposure is shown in FIG. 6. Cell survival decreased from 100% to 70% and from 95% to 72% for doxorubicin-only treated cells in the dark and upon light irradiation, respectively. A very low decrease (average of 2-5%) in cell survival was observed with the doxorubicin-treated cells upon light irradiation compared to unirradiated cells. PS-treated cells (at 100 nM) in the dark showed no cytotoxicity (FIG. 7A). This is the same data shown in FIG. 4A, and included here for comparison. Co-treatment of TNBC cells with PS (100 nM) and doxorubicin (500 nM) for 24 h without light exposure resulted in an average of 63% cell survival (51% lowest for CBTN and 75% highest for CPA) compared to doxorubicin-only-treated cells with 81% survival. In the absence of light, only the cytotoxic effect of doxorubicin was involved in cell destruction.

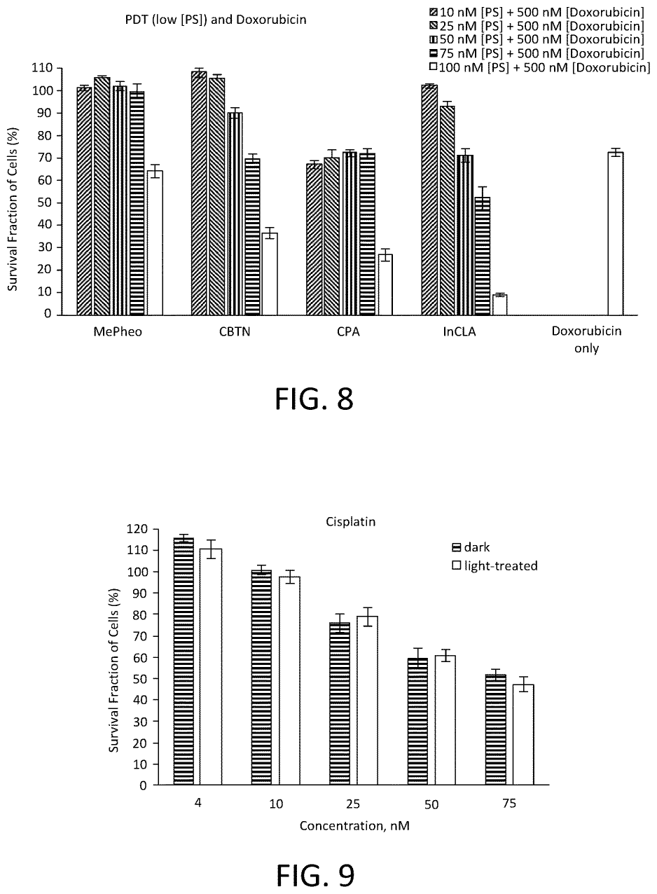

Upon light exposure of co-treated cells with 500 nM doxorubicin (FIG. 7B), InCLA (at 100 nM) was still the most phototoxic, with an observed 9% cell survival, followed by CBTN and CPA having 35% and 37% cell viability, respectively. InCLA still overwhelms the effect of doxorubicin at this concentration. CBTN and CPA at 100 nM showed better photodynamic effect when co-treated with doxorubicin than PS-treatment alone. CBTN decreased from 82% to 36%, while CPA went from 75% to 27%, with an average cell viability reduction of 47% for both PSs. Lower concentrations (10-75 nM) of InCLA with 500 nM doxorubicin did not show additive nor synergistic effect upon co-treatment (FIG. 8). Combination therapy was observed to be better than monotherapy alone depending on the PSs used.

iii) PDT and Cisplatin

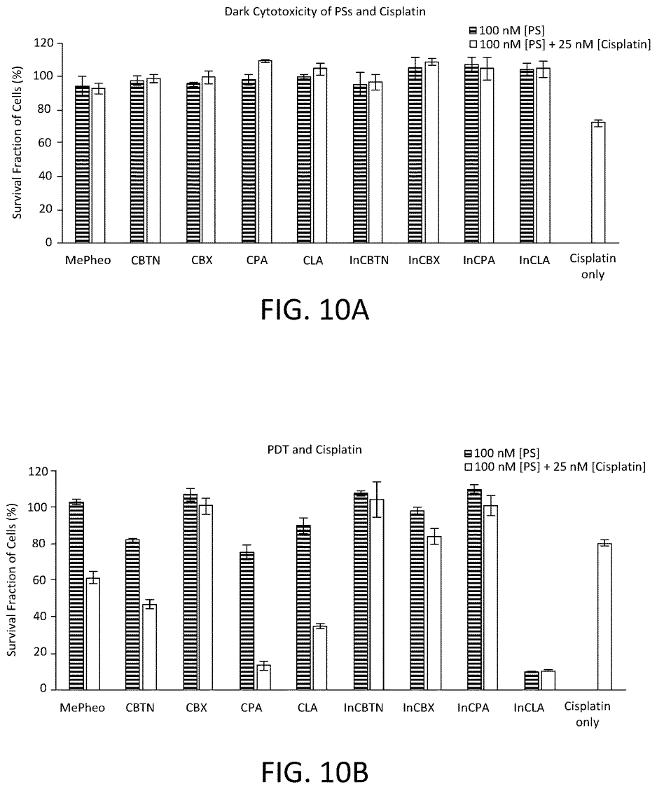

Cytotoxicity of cisplatin at varying concentrations of 4-75 nM in TNBC cells in the dark and upon light exposure is shown in FIG. 9. Cell survival decreased from >100% to 52% and from >100% to 47% for cisplatin-only treated cells in the dark and upon light irradiation, respectively. A very low decrease (average of 3%) in cell survival was observed with the cisplatin-treated cells upon light irradiation compared to unirradiated cells. PS-treated cells (at 100 nM) in the dark showed no cytotoxicity (FIG. 10A), which represents the same data shown in FIG. 8A for comparison. Co-treatment of TNBC cells with PS (100 nM) and cisplatin (25 μM) for 24 h without light exposure did not result in any significant alteration in cellular viability compared to cisplatin-only-treated cells with 72% survival (FIG. 10A). In the absence of light, the presence of cisplatin in the TNBC cells caused no effect.

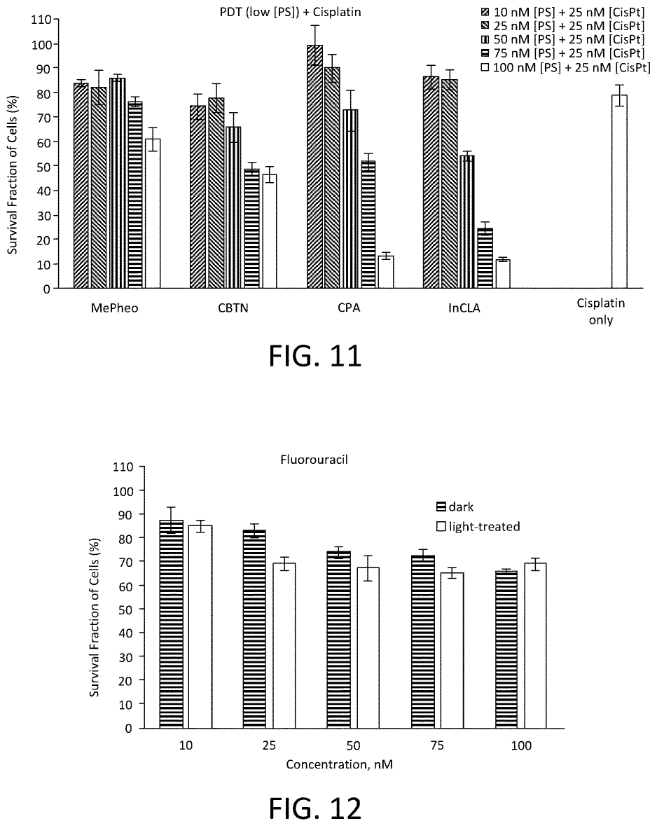

Upon light exposure of co-treated cells with 25 μM cisplatin (FIG. 10B), InCLA (at 100 nM) remains as the most phototoxic with an observed 10% cell survival, followed by CPA and CLA having 13% and 35% cell viability, respectively. InCLA also overwhelmed the effect of cisplatin at this concentration. CPA and CLA at 100 nM showed better photodynamic effect when co-treated with cisplatin than PS-treatment alone. CPA decreased from 75% to 13%, while CLA went from 90% to 35%, with an average decline of 59% cell survival for both PSs Except for InCLA, a decrease in cell viability of TNBC cells ranges from 62% to 3% upon co-treatment with 25 μM cisplatin compared to PS-treatment alone at 100 nM. There is an average of 28% decrease between combination therapy compared to PS treatment alone (FIG. 4B). However, when compared to cisplatin-treated cell viability, the effect of added cisplatin caused a reduced cell survival only in MePheo-, CBTN-, CPA-, and CLA-co-treated TNBC cells. At lower concentrations of selected PSs (MePheo, CBTN, CPA and InCLA) at 50 nM co-treated with 25 μM cisplatin, a reduced cell survival ranging from 28% to 11% is observed with an average of 23% as shown in FIG. 11 compared to PS-treated only cells in FIG. 2. Combination therapy with cisplatin was observed to be better than monotherapy alone depending on the PSs used.

### iv) PDT and Fluorouracil

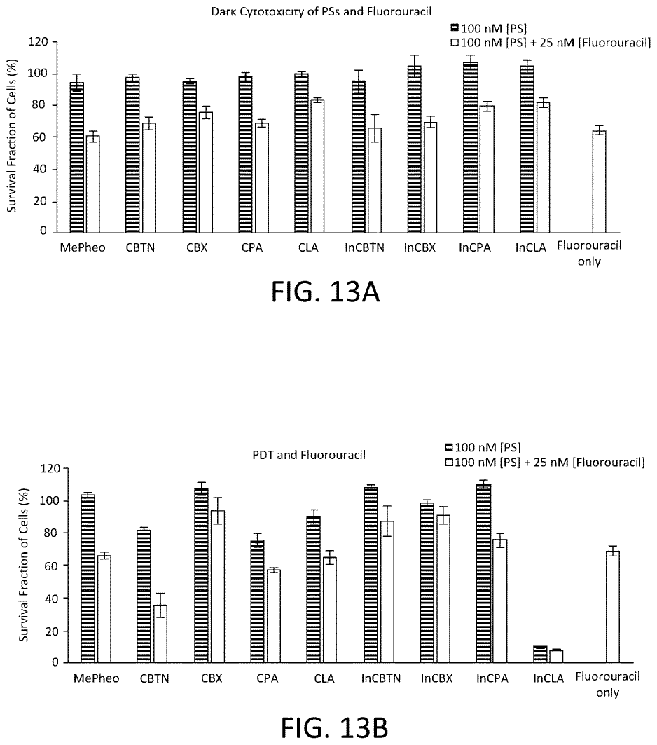

Cytotoxicity of fluorouracil at varying concentrations of 10-100 nM in TNBC cells in the dark and upon light exposure is shown in FIG. 12. Cell survival decreased from 87% to 65% and from 85% to 68% for fluorouracil-only treated cells in the dark and upon light irradiation, respectively. A decrease in cell survival with an average of 9% was observed with the fluorouracil-treated cells upon light irradiation compared to unirradiated cells. PS-treated cells (at 100 nM) in the dark showed no cytotoxicity (FIG. 13A), which represents the same data shown in FIG. 4A for comparison. Co-treatment of TNBC cells with PS (100 nM) and fluorouracil (25 μM) for 24 hours without light exposure resulted in an average of 73% cell survival (61% lowest for MePheo and 84% highest for CLA) compared to fluorouracil-only-treated cells with 64% survival. In the absence of light, only the cytotoxic effect of fluorouracil is involved in cell destruction (FIG. 13A).

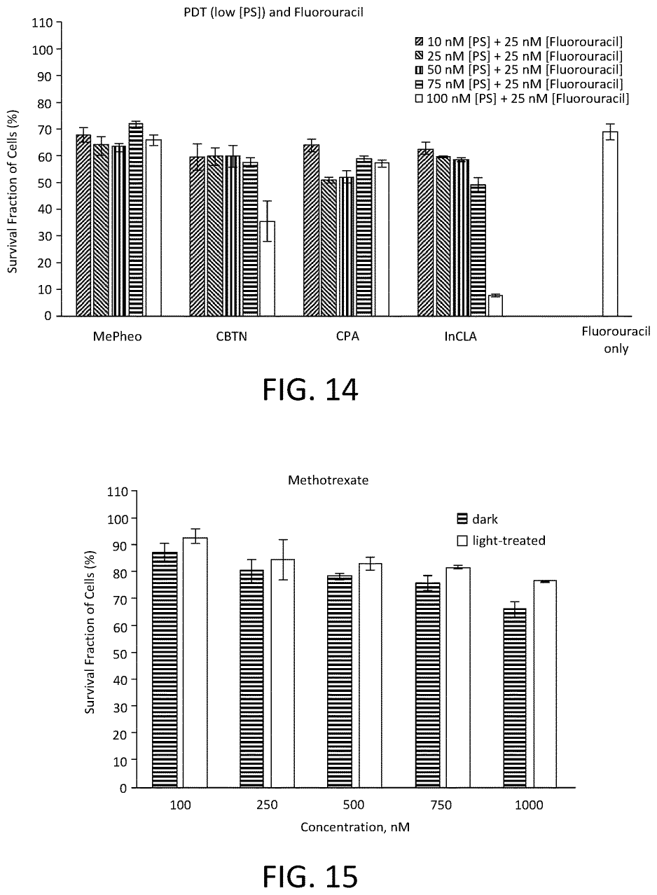

Upon light exposure of co-treated cells with 25 μM fluorouracil (FIG. 13B), InCLA (at 100 nM) still remains as the most phototoxic with an observed 8% cell survival, followed by CBTN and CPA having 36% and 57% cell viability, respectively. InCLA also overwhelmed the effect of fluorouracil at this concentration. CBTN and CPA at 100 nM showed better photodynamic effect when co-treated with fluorouracil than PS-treatment alone. CBTN decreased from 81% to 36%, while CPA went from 75% to 57%, with an average decline of 31% cell survival for both PSs. Except for InCLA, a decrease in cell viability of TNBC cells ranges from 46% to 7% upon co-treatment with 25 μM fluorouracil compared to PS-treatment alone at 100 nM. There is an average of 22% decrease between combination therapy compared to PS treatment alone (FIG. 4B). However, when compared to fluorouracil-treated cell viability, the effect of added fluorouracil caused a reduced cell survival only in CBTN-, and CPA-co-treated TNBC cells. At lower concentrations of selected PSs, only CBTN at 100 nM co-treated with 25 μM fluorouracil showed a significant cell viability reduction from 82% to 36% with a difference of 46% as shown in FIG. 13B (or FIG. 14) compared to PS-treated only cells in FIG. 2. CBTN as PS in combination with fluorouracil was observed to be the best compared to monotherapy alone and with the other PSs used.

### V) PDT and Methotrexate

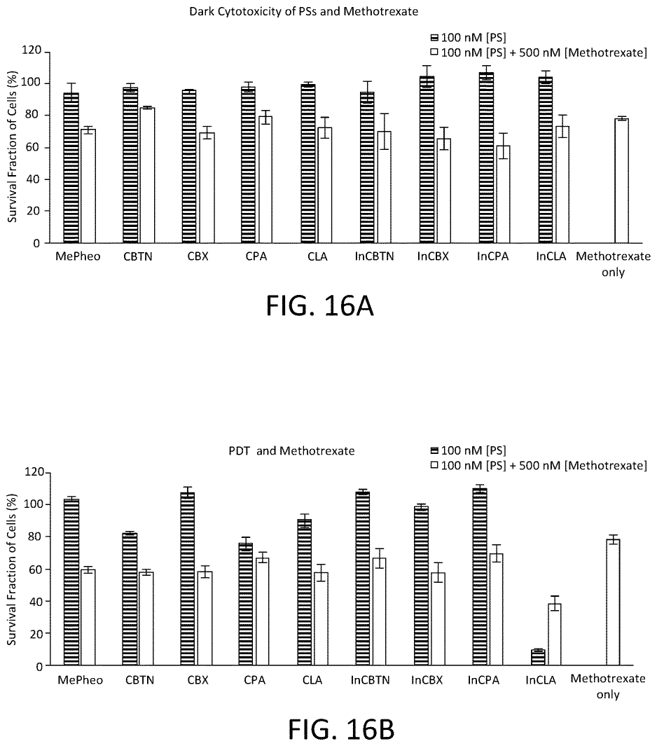

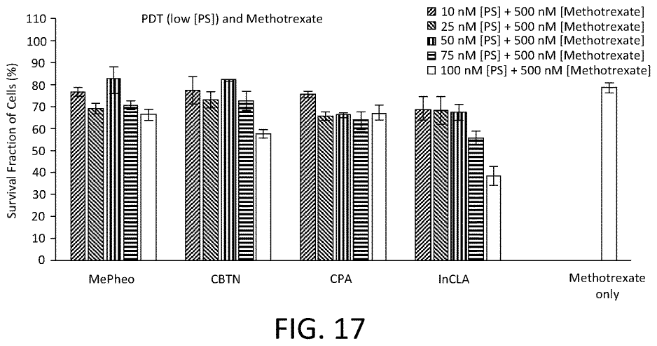

Cytotoxicity of methotrexate at varying concentrations of 100-1000 nM in TNBC cells in the dark and upon light exposure is shown in FIG. 15. Cell survival decreased from 87% to 66% and from 93% to 66% for methotrexate-only treated cells in the dark and upon light irradiation, respectively. An increase (average of 6%) in cell survival is observed with the methotrexate-treated cells upon light irradiation compared to unirradiated cells. PS-treated cells (at 100 nM) in the dark showed no cytotoxicity (FIG. 16A), which represents the same data shown in FIG. 4A for comparison. Co-treatment of TNBC cells with PS (100 nM) and methotrexate (500 nM) for 24 hours without light exposure resulted in an average of 72% cell survival (61% lowest for InCPA and 85% highest for CBTN) compared to methotrexate-only-treated cells with 78% survival. In the absence of light, only the cytotoxic effect of methotrexate was involved in cell destruction (FIG. 16A).

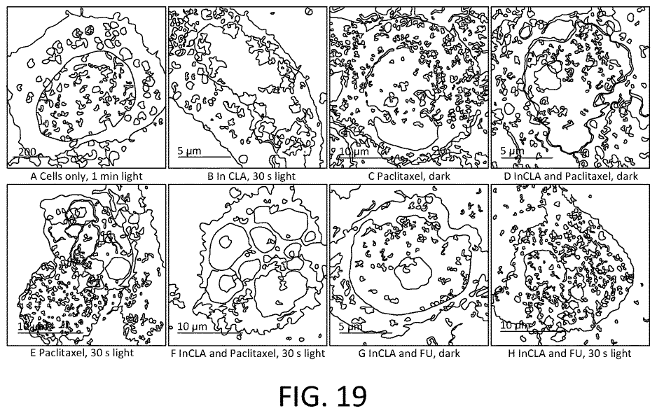

Upon light irradiation of co-treated cells with 500 nM methotrexate (FIG. 13B), InCLA (at 100 nM) still remains as the most phototoxic among the PSs tested with an observed 39% cell survival, followed by CLA, then by CBTN, CBX, and InCBX having the same 57-58% cell viability. However, InCLA was observed to cause an enhanced cell viability at this concentration. At lower concentrations of selected PSs, only CPA at 50 nM co-treated with methotrexate (500 nM) showed a significant cell viability reduction from 99% to 66% with a difference of 33%. MePheo and CBTN were observed to cause a difference of only 10% cell survival compared to PS treatment alone (FIG. 19). CPA as PS in combination with methotrexate appeared to be the best compared to monotherapy alone and with the other PSs used.

### Cell Viability Summary and Combination Index

Cell inhibition in TNBC cells is based on the cell survival (MTT) assay of the binary therapy, blending the effect of PDT and chemotherapeutic agents. Table 1 tabulates the percent inhibition of TNBC cells treated with eight synthesized PSs (CBTN, CBX, CPA, CLA, and their corresponding indium complexes) and the starting compound MePheo at 100 nM, and simultaneously treated with paclitaxel (50 nM), doxorubicin (500 nM), cisplatin (25 mM), fluorouracil (25 mM), or methotrexate (500 nM). MePheo serves as a control compound. TNBC cells were then exposed to light after binary treatment for 24 hours.

TABLE 1
shows cell inhibition (%) of TNBC cells treated with photosensitizers
(100 nM) and chemotherapeutic drugs for 24 hours, followed by
light irradiation (light dose of 0.96 J cm −2). Chemotherapeutic
drugs: paclitaxel (50 nM), doxorubicin (500 nM), cisplatin (25
μM), fluorouracil (25 μM); and, methotrexate (500 nM).

| Photosen- | Pacli- | | | | |
|---|---|---|---|---|---|
| sitizer | taxel | Doxorubicin | Cisplatin | Fluorouracil | Methotrexate |
| MePheo | 55 | 36 | 39 | 34 | 40 |
| CBTN | 65 | 64 | 53 | 64 | 42 |
| CBX | 48 | 30 | 0 | 6 | 42 |
| CPA | 63 | 73 | 87 | 43 | 33 |
| CLA | 45 | 33 | 65 | 35 | 43 |
| InCBTN | 45 | 25 | 0 | 13 | 33 |
| InCBX | 45 | 41 | 16 | 9 | 42 |
| InCPA | 51 | 27 | 0 | 25 | 30 |
| InCLA | 91 | 91 | 90 | 92 | 61 |

The order of inhibition for the nine PSs tested in this study from the most potent to the least potent chemophotodynamic efficacy is provided as follows:

For PSs and paclitaxel combination:
InCLA>>CBTN>CPA>MePheo>InCPA>CBX>CLA, InCBTN, InCBX
   For PSs and doxorubicin combination:
InCLA>>CPA>CBTN>InCBX>MePheo>CLA>CBX>InCPA>InCBTN
   For PSs and cisplatin combination:
InCLA>CPA>CLA>CBTN>MePheo>InCBX>CBX, InCBTN, InCPA
   For PSs and methotrexate combination:
InCLA>>CLA>CBTN, CBX, InCBX>MePheo>CPA, InCBTN>InCPA
   For PSs and fluorouracil combination:
InCBTN, InCPA>InCBX>CBTN>CPA>CLA>MePheo>InCLA>CBX

Based on these data of the PSs at 100 nM concentration, InCLA exhibited the most potency either alone or in combination, specifically with paclitaxel, doxorubicin, cisplatin, and fluorouracil. Following InCLA were CPA and CBTN having the best chemophotodynamic efficacy among the synthesized PSs tested. InCLA, CPA, and CBTN showed better activity against TNBC cells than the starting compound MePheo. The best combination for CBTN is with paclitaxel followed by doxorubicin or fluorouracil, while for CPA, cisplatin is the best, next doxorubicin, then paclitaxel.

The combination index (CI) for the binary treatment of TNBC cells with a PS and a chemotherapeutic drug was calculated based on the Chou-Talalay method using CompuSyn software. Interaction dynamics of multiple entities resulted in a combination index equation, and this computer simulation offered an automatic quantitative determination of synergism (CI<1), additive (CI=1), and antagonism (CI>1). The unified dynamics algorithm uses a minimum of dose-data points to fit the general MAL theory.

TABLE 2
shows combination index (CI) values of binary therapy
(selected PSs and chemotherapeutic drugs) and the corresponding
synergism vs additive vs antagonism effects.

| | Pacli- | | | | |
|---|---|---|---|---|---|
| PSs | taxel | Doxorubicin | Cisplatin | Fluorouracil | Methotrexate |
| 50 nM | 50 nM | 500 nM | 25 μM | 25 μM | 500 μM |
| MePheo | 13.27 | 6.37 | 6.10 | 17.96 | 7.79 |
| | (VSA) | (SA) | (SA) | (VSA) | (SA) |
| CBTN | 0.74 | 2.44 | 0.99 | 0.48 | 1.57 |
| | (MS) | (A) | (NA) | (S) | (A) |
| CPA | 0.41 | 1.52 | 1.30 | 0.45 | 0.50 |
| | (S) | (A) | (MA) | (S) | (S) |
| InCLA | 0.25 | 1.91 | 1.09 | 0.94 | 1.21 |
| | (SS) | (MA) | (NA) | (NA) | (MA) |

CI = 1 (additive); CI < 1 (synergism); CI > 1 (antagonism).
VSS (<0.1), very strong synergism; SS (0.1-0.3), strong synergism; S (0.3-0.7), synergism; MS (0.7-0.85), moderate synergism; StS (0.85-0.90), slight synergism; NA (0.9-1.10), nearly additive; StA (1.10-1.20), slight antagonism; MA (1.20-1.45), moderate antagonism; A (1.45-3.3), antagonism; SA (3.3-10), strong antagonism; VSA (>10), very strong antagonism.
Concentrations: PSs = 50 nM; paclitaxel = 50 nM; doxorubicin = 500 nM; cisplatin = 25 μM; fluorouracil = 25 μM; methotrexate = 500 nM.

The CI values for PSs selected based on the best biological activity to inhibit the growth of TNBC cells in combination with the known chemotherapeutic entity used in this study are listed in Table 2 as calculated using CompuSyn software. The synthesized PSs proved to be better than the starting compound MePheo and confirmed the results in the cell viability assay. The combination of InCLA and paclitaxel showed the strongest synergism with the lowest CI value of 0.25. Other combinations in the table range from synergistic to nearly additive, with CI values ranging from 0.41-1.09. The calculated CI values are based on 50 nM concentration for the PSs, 50 nM paclitaxel, 500 nM doxorubicin, 25 μM cisplatin, 25 μM fluorouracil, and 500 nM methotrexate.

Ranking the combinations based on the CI values in decreasing order:
InCLA: paclitaxel>fluorouracil>cisplatin
CPA: paclitaxel>fluorouracil>methotrexate
CBTN: fluorouracil>paclitaxel>cisplatin

Based on the low nanomolar dosage used for the binary therapy, the best chemophotodynamic combination treatment against TNBC in decreasing order is the following:
InCLA+paclitaxel>CPA+paclitaxel>CBTN+paclitaxel

### Microscopy Studies

### i) Fluorescence Microscopy

PDT can initiate several forms of cell death pathways that include apoptosis, autophagy and/or necrosis. Morphological characteristics of apoptosis can be identified under light microscopy, and include cell shrinkage, chromatin condensation, nuclear fragmentation, blebbing of the cytoplasmic membrane, loss of adhesion and cellular volume, and, finally, the formation of apoptotic bodies. Autophagy (or self-eating) is a conserved cellular degradation process that eliminates molecules and subcellular elements, including nucleic acids, proteins, lipids, and organelles, via lysosome-mediated degradation, maintaining and promoting homeostasis, differentiation, development, and survival, and preventing nutritional, metabolic, and infection-mediated stresses. Necrotic cell death is characterized morphologically by generalized swelling of cell membranes, often accompanied by some condensation of nuclear chromatin, rupture of plasma membrane, and an irregular DNA degradation pattern.

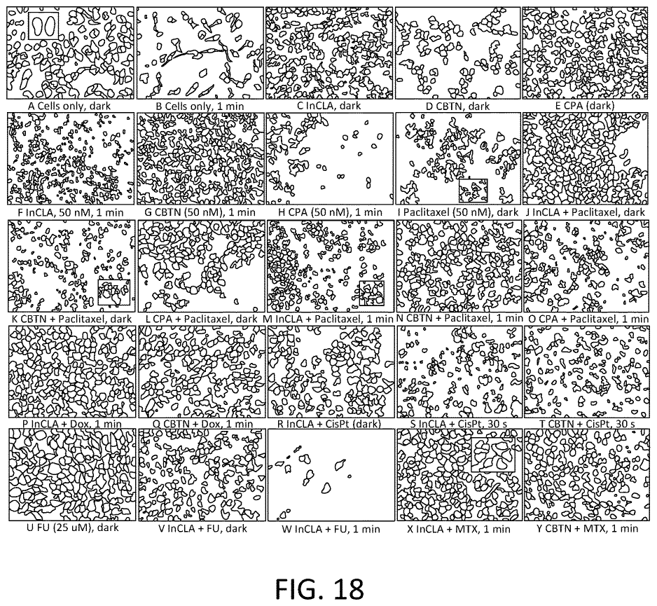

To determine the preferred cell death pathway for the binary chemophotodynamic therapy, tracking the hallmarks of apoptosis or necrosis was accomplished using a nuclear stain to monitor chromatin condensation and cellular contraction. Fluorescence microscopy images of TNBC cells stained with Hoechst 33258 nuclear stain are shown in FIG. 18. TNBC cells are spindle-shaped and nearly round with intact cytoplasm, diffused chromatin, distinct cellular membrane, and large oval nuclei staining dark blue with the nuclear stain (FIG. 18A-B). PS-treated cells in the dark (FIG. 18C-E) exhibit morphology similar to the untreated and irradiated sham control (FIG. 18A-B). InCLA- and CPA-treated cells clearly showed reduced cellular volume and nuclear density, but not as obvious in CBTN-treated cells at 50 nM concentration 24-hour treatment followed by light exposure (FIG. 18F-H). For paclitaxel-treated cells (FIG. 181), and cells with PSs (InCLA, CBTN and CPA) co-treated with paclitaxel (FIG. 18M-O), shrunk cells and chromatin fragmentation are evident, showing apoptosis as the preferred pathway. No significant difference was observed between sham control cells and cells subjected to combination treatment with InCLA (50 nM) or CBTN (50 nM) with doxorubicin (500 nM) (FIG. 19P-Q). With combined cisplatin and InCLA in the dark, very few shrunk cells can be seen but not significant (FIG. 18R). However, when exposed to light for 30 seconds (light dose 0.48 J/cm²), a significant number of shrunk cells and chromatin condensation can be seen (FIG. 18S-T) for InCLA- or CBTN- and cisplatin-cotreated cells. Cells treated with fluorouracil at 25 μM cells in the dark did not affect the cellular morphology (FIG. 18U), as well as with InCLA with fluorouracil (FIG. 18V). However, upon light irradiation, an extremely reduced cell density is apparent (FIG. 18W). Very few cells exhibit chromatin condensation nor any morphological changes indicative of apoptosis for cells treated with InCLA or CBTN co-treated with methotrexate followed by irradiation (FIG. 18X-Y).

Experimental results from the fluorescence microscopy technique indicated that the preferred mode of cell death is apoptosis when our synthesized PSs (InCLA, CBTN, and CPA) were co-treated with paclitaxel or cisplatin. No evidence of cell swelling suggesting necrosis is observed. The combination of PSs with fluorouracil showed loss of cell viability as indicated by reduced cell density, possibly due to loss of cell adhesion and contact with neighboring cells, but apoptosis seems to be somewhat preferred more than necrosis.

### ii) Transmission Electron Microscopy Studies

Apoptosis, necrosis, and autophagy are now recognized as the most common mechanisms of cell deletions characterized by peculiar morphology of a physiologically occurring cell death. Transmission electron microscopy (TEM) provides a detailed depiction of these phenomena and, due to its high resolution, is one of the most powerful morphological technique to visualize the inner cellular and organelle ultrastructural alterations under physiological and pathological conditions (Rembiałkowska N et al., Applied Sciences, 2020, 10(8), 2765). Histopathology can recognize differences in these three pathways.

Transmission electron micrographs of TNBC cells clearly differentiated distinct cellular compartments, in particular, of nuclear components and plasma membrane during treatment as shown in FIG. 19. A typical normal organelle morphology with smooth-contoured cellular plasma membrane is depicted in TNBC cells irradiated with light without treatment with PS nor chemotherapeutic agent (FIG. 19A). Chromatin shows an abnormal pattern with elongated nuclear envelope and numerous clumps of shredded DNA around the inner nuclear membrane in InCLA-treated and irradiated cells (FIG. 19B) indicative of the early phase of apoptosis. A lower light dose of 0.48 J/cm² was applied to monitor the modifications that cells undergo during PDT treatment. Irregularly shaped nuclear envelope in paclitaxel-treated and in InCLA co-treated cells in the dark as shown in FIGS. 19C-19D also demonstrates cellular changes upon treatment. The effect of paclitaxel alone without light was evident in the loss of nuclear circularity and formation of chromatin condensation. When paclitaxel-treated (FIG. 19E) and PS co-treated (FIG. 19F) TNBC cells were illuminated, smaller nuclei in multi-nucleated cells indicating chromosomal condensation were observed. Multinucleation was recognized as histopathological evidence of cells that are compromised, characterized with smaller nuclear size and smaller nuclear circularity, and are associated with certain diseases. The cytoplasm in paclitaxel-treated cells (FIG. 19E) had split and fragmented with many atypical vesicular components in the cytosolic compartment. Several ultrastructural features typical in apoptotic cell death were evident in TNBC cells co-treated with InCLA and paclitaxel in the presence of light (FIG. 19F), such as cellular surface blebbing and overlapping disintegrated nuclear pores. In co-treated cells with InCLA and fluorouracil in the dark (FIG. 19G), the chromatin is abnormally condensed into a large central nuclear clump (stained dark) with chromatin marginalization and irregularly-shaped cellular membrane depicting the timeline during the early phase of the apoptotic pathway. In the presence of light (FIG. 19G), large vacuoles and vesicles in the cytoplasm are observed.

TEM images confirmed results from fluorescence microscopy that apoptosis seems to be the preferred mode of cell death during chemophotodynamic treatment. However, TEM data appeared to suggest that mitotic catastrophe and some autophagy also occurred, similar to another study conducted on the effect of paclitaxel in human gastric adenocarcinoma AGS cell line in which apoptosis, autophagy, and mitotic catastrophe were triggered by paclitaxel. Aside from the most common mode of cell death mechanistic pathways such as apoptosis (manifested by volume reduction of nucleus and cytoplasmic or cell shrinkage), necrosis (cytoplasmic or mitochondrial swelling and plasma membrane rupture), and autophagy (accumulation of cytosolic vacuoles and membranes), mitotic catastrophe was also a cell death mechanism triggered by aberrant or dysregulated mitosis. Morphological markers of mitotic catastrophe are micronucleation or multinucleation, in which multinucleated cells are formed from the clusters of mis-segregated and uncondensed chromosomes. Hence, multiple cell death mechanisms, to some degree, were activated during combination therapy.

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range.

The term “substantially” can allow for a degree of variability in a value or range, for example, within 90%, within 95%, or within 99% of a stated value or of a stated limit of a range.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the terms “a,” “an,” or “the” are used to include one or more than one unless the context clearly dictates otherwise. The term “or” is used to refer to a nonexclusive “or” unless otherwise indicated. In addition, the phraseology or terminology employed herein, and not otherwise defined, is for the purpose of description only and not of limitation. Any use of section headings is intended to aid the reading of the document and is not to be interpreted as limiting. Further, information that is relevant to a section heading may occur within or outside of that particular section. The terms “including” and “having” are defined as comprising (i.e., open language).

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

In addition, any of the embodiments described in the following clause list are considered to be part of the invention.
A. A pharmaceutical combination comprising (i) a chlorin-vitamin conjugate or its pharmaceutically acceptable salt, hydrate, or metal complex and (ii) at least one chemotherapeutic agent, wherein the chlorin-vitamin conjugate or its pharmaceutically acceptable salt, hydrate, or metal complex is a photosensitizer.
B. The pharmaceutical combination of clause A, wherein the chlorin-vitamin conjugate is a chlorin lipoic acid conjugate (CLA), a chlorin biotin conjugate (CBTN), a chlorin pantothenic acid conjugate (CPA), a chlorin bexarotene conjugate (CBX), a chlorin desthiobiotin conjugate (CDBTN), a chlorin biocytin conjugate (CBC), or a combination of two or more thereof.
C. The pharmaceutical combination of clause A or B, wherein a metal in the metal complex of the chlorin-vitamin conjugate is zinc, indium, palladium, or platinum.
D. The pharmaceutical combination of clause A, wherein the chemotherapeutic agent is selected from the group consisting of paclitaxel, docetaxel, doxorubicin, cisplatin, fluorouracil, methotrexate, paraplatin, cyclophosphamide, epirubicin, gemcitabine, eribulin, ixabepilone, mutamycin, vinorelbine, capecitabine, daunorubicin, idarubicin, mitoxantrone, vinblastine, vincristine, vinorelbine, actinomycin D, bleomycin, daunomycin, and a combination of two or more thereof.
E. The pharmaceutical combination of clause D, wherein the chemotherapeutic agent is paclitaxel, doxorubicin, cisplatin, fluorouracil, methotrexate, or a combination of two or more thereof.
F. A pharmaceutical composition comprising therapeutically effective amounts of (i) and (ii) of the pharmaceutical combination of any one of clauses A-E and a pharmaceutically acceptable carrier, diluent, or excipient.
G. The pharmaceutical composition of clause F, which is comprised of nanoparticles.
H. A combination of pharmaceutical compositions comprising (i) a pharmaceutical composition comprising a therapeutically effective amount of the chlorin-vitamin conjugate or its pharmaceutically acceptable salt, hydrate, or metal complex of any one of clauses A-C and (ii) a pharmaceutical composition comprising a therapeutically effective amount of the at least one chemotherapeutic agent of any one of clauses A, D and E, wherein (i) and (ii) are independently formulated to be administered by the same or different routes.
I. The combination of pharmaceutical compositions of clause H, wherein either one or both of (i) and (ii) are comprised of nanoparticles.
J. The combination of pharmaceutical compositions of clause H or I, wherein (i) and (ii) are independently formulated to be administered intravenously, topically, or orally.
K. A method for treating a patient for cancer, which method comprises administering to the patient (i) a therapeutically effective amount of a chlorin-vitamin conjugate or its pharmaceutically acceptable salt, hydrate, or metal complex and (ii) a therapeutically effective amount of at least one chemotherapeutic agent, wherein the chlorin-vitamin conjugate or its pharmaceutically acceptable salt, hydrate, or metal complex is a photosensitizer, whereupon the patient is treated for cancer.
L. The method of clause K, wherein (i) and (ii) are formulated as a single pharmaceutical composition or as separate pharmaceutical compositions, which can be administered simultaneously or sequentially by the same or different routes.
M. The method of claim K or L, wherein (i) and (ii) are administered intravenously, topically, or orally.
N. The method of any one of clauses K-M, wherein the therapeutically effective amount of (i) is from about 30 nM to about 100 nM.
O. The method of any one of clauses K-N, wherein the therapeutically effective amount of (ii) is from about 25 nM to about 25 μM.
P. The method of clause K, wherein the cancer is selected from the group consisting of head and neck cancer, breast cancer, prostate cancer, lung cancer, liver cancer, gynecological cancer, cervical cancer, brain cancer, melanoma, colorectal cancer, bladder cancer, ovarian cancer, and gastrointestinal cancer.
Q. The method of clause K, wherein the cancer is breast cancer.
R. The method of clause Q, wherein the breast cancer is triple-negative.
S. The method of any one of clauses K-R, wherein (i) and (ii) have a synergistic effect.

1. A pharmaceutical combination comprising (i) a chlorin-vitamin conjugate or its pharmaceutically acceptable salt, hydrate, or metal complex and (ii) at least one chemotherapeutic agent, wherein the chlorin-vitamin conjugate or its pharmaceutically acceptable salt, hydrate, or metal complex is a photosensitizer.

2. The pharmaceutical combination of claim 1, wherein the chlorin-vitamin conjugate is a chlorin lipoic acid conjugate (CLA), a chlorin biotin conjugate (CBTN), a chlorin pantothenic acid conjugate (CPA), a chlorin bexarotene conjugate (CBX), a chlorin desthiobiotin conjugate (CDBTN), a chlorin biocytin conjugate (CBC), or a combination of two or more thereof.

3. The pharmaceutical combination of claim 1 or 2, wherein a metal in the metal complex of the chlorin-vitamin conjugate is zinc, indium, palladium, or platinum.

4. The pharmaceutical combination of claim 1, wherein the chemotherapeutic agent is selected from the group consisting of paclitaxel, docetaxel, doxorubicin, cisplatin, fluorouracil, methotrexate, paraplatin, cyclophosphamide, epirubicin, gemcitabine, eribulin, ixabepilone, mutamycin, vinorelbine, capecitabine, daunorubicin, idarubicin, mitoxantrone, vinblastine, vincristine, vinorelbine, actinomycin D, bleomycin, daunomycin, and a combination of two or more thereof.

5. The pharmaceutical combination of claim 4, wherein the chemotherapeutic agent is paclitaxel, doxorubicin, cisplatin, fluorouracil, methotrexate, or a combination of two or more thereof.

6. A pharmaceutical composition comprising therapeutically effective amounts of (i) and (ii) of the pharmaceutical combination of any one of claims 1-5 and a pharmaceutically acceptable carrier, diluent, or excipient.

7. The pharmaceutical composition of claim 6, which is comprised of nanoparticles.

8. A combination of pharmaceutical compositions comprising (i) a pharmaceutical composition comprising a therapeutically effective amount of the chlorin-vitamin conjugate or its pharmaceutically acceptable salt, hydrate, or metal complex of any one of claims 1-3 and (ii) a pharmaceutical composition comprising a therapeutically effective amount of the at least one chemotherapeutic agent of any one of claims 1, 4 and 5, wherein (i) and (ii) are independently formulated to be administered by the same or different routes.

9. The combination of pharmaceutical compositions of claim 8, wherein either one or both of (i) and (ii) are comprised of nanoparticles.

10. The combination of pharmaceutical compositions of claim 8 or 9, wherein (i) and (ii) are independently formulated to be administered intravenously, topically, or orally.

11. A method for treating a patient for cancer, which method comprises administering to the patient (i) a therapeutically effective amount of a chlorin-vitamin conjugate or its pharmaceutically acceptable salt, hydrate, or metal complex and (ii) a therapeutically effective amount of at least one chemotherapeutic agent, wherein the chlorin-vitamin conjugate or its pharmaceutically acceptable salt, hydrate, or metal complex is a photosensitizer, whereupon the patient is treated for cancer.

12. The method of claim 11, wherein (i) and (ii) are formulated as a single pharmaceutical composition or as separate pharmaceutical compositions, which can be administered simultaneously or sequentially by the same or different routes.

13. The method of claim 11 or 12, wherein (i) and (ii) are administered intravenously, topically, or orally.

14. The method of any one of claims 11-13, wherein the therapeutically effective amount of (i) is from about 30 nM to about 100 nM

15. The method of any one of claims 11-14, wherein the therapeutically effective amount of (ii) is from about 25 nM to about 25 □M.

16. The method of claim 11, wherein the cancer is selected from the group consisting of head and neck cancer, breast cancer, prostate cancer, lung cancer, liver cancer, gynecological cancer, cervical cancer, brain cancer, melanoma, colorectal cancer, bladder cancer, ovarian cancer, and gastrointestinal cancer

17. The method of claim 11, wherein the cancer is breast cancer.

18. The method of claim 17, wherein the breast cancer is triple-negative.

19. The method of any one of claims 11-18, wherein (i) and (ii) have a synergistic effect.
